(19) United States
(12) Patent Application Publication
(10) Pub. No.: US 2019/0060281 A1
(43) Pub. Date: Feb. 28, 2019

(54) COMPOSITIONS AND TREATMENT METHODS FOR DIAPER DERMATITIS AND SKIN IRRITATION

(71) Applicant: Jessica S. WHITTLE, Hixson, TN (US)

(72) Inventor: Jessica S. WHITTLE, Hixson, TN (US)

(21) Appl. No.: 16/108,212

(22) Filed: Aug. 22, 2018

Related U.S. Application Data
(60) Provisional application No. 62/549,548, filed on Aug. 24, 2017.

(51) Int. Cl. A61K 31/4174; A61K 38/12; A61K 47/02; A61K 47/06; A61K 47/44; A61K 8/92; A61K 8/31; A61K 8/26; A61Q 17/00; A61P 17/04; A61K 9/00

(52) U.S. Cl. CPC A61K 31/4174; A61K 38/12; A61K 47/02; A61K 47/06; A61K 47/44; A61K 9/0014; A61K 8/31; A61K 8/26; A61Q 17/00; A61P 17/04; A61K 8/925

(57) ABSTRACT

Disclosed herein are topical compositions and treatment methods for protecting the skin and alleviating symptoms of dermatitis, including “diaper rash”, and skin irritation caused by nonspecific dermatitis conditions and those associated with increased bacterial and/or yeast growth. The compositions may include petroleum jelly, lanolin, aluminum hydroxide, mineral oil, miconazole and bacitracin. The compositions may be delivered topically and formulated in ointments, creams, and lotions.

### CROSS-REFERENCE TO RELATED APPLICATION(S)

The present patent application claims priority to U.S. Provisional Application No. 62/549,548, filed Aug. 24, 2017, and entitled “Composition for the Treatment of Diaper Dermatitis and Skin Irritation”, the entire disclosure of which is incorporated herein by reference.

### FIELD OF THE DISCLOSURE

The present disclosure relates generally to topical compositions used to protect the skin and treat the skin condition commonly known as “diaper rash” or “diaper dermatitis”. More specifically, the present disclosure relates to unique formulations of a topical ointment skin protectant and treatment for dermatitis. The compositions and treatment methods may further include using miconazole for the killing of yeast and/or bacitracin for antibacterial action.

### BACKGROUND OF THE DISCLOSURE

Skin may encounter harmful elements on a daily basis, including but limited to, moisture, irritants such as urine and feces, bacteria, fungus, and allergens. “Diaper rash” or “diaper dermatitis” is a skin condition prevalent amongst infants and commonly associated with increased bacteria and/or yeast growth.

Many topical skin ointments exist and are commonly used to protect and treat a variety of skin types and conditions. Many over the counter creams for the treatment of “diaper dermatitis” have focused on providing hydrophobic barriers, breaking down excess ammonia, controlling pH levels, and using zinc oxide.

U.S. Pat. No. 3,567,820 discloses using cation-exchange resins to absorb ammonia irritants that may remain despite the actions of germicide components to eliminate ammonia-forming organisms.

U.S. Pat. No. 4,996,238 discloses a skin protective composition containing aliphatic waxes and hydrophobic silicones to enhance water repulsion and skin conditioning. This composition does not include any water or pH buffers.

U.S. Pat. No. 5,091,193 discloses using zinc oxide and an anti-enzyme to treat or prevent diaper rash.

U.S. Pat. No. 5,362,488 discloses a water-based skin protectant with a buffering system effective in acidic conditions.

U.S. Pat. No. 5,762,945 discloses using nystatin powder with zinc oxide to eliminate bacterial and yeast generated irritants.

U.S. Pat. No. 6,805,875 discloses using a pH buffered ointment to control some forms of diaper rash caused by acidic irritants.

None of these however, focus on the synergistic effect of combining the actions of a buffered barrier ointment with bacitracin and/or miconazole to control bacterial and/or yeast overgrowth.

Thus, what is still needed in the art is a composition and method for protecting skin and treating dermatitis that targets yeast and bacterial overgrowth as well as acting as a pH buffered skin protectant and having a desiccant action to balance skin surface dryness with internal skin moisturization.

### BRIEF SUMMARY OF THE DISCLOSURE

The present disclosure addresses the issues associated with treating dermatitis using unique formulations of a topical ointment skin protectant and treatment for dermatitis, including but not limited to, cases of dermatitis associated with candida infection and/or mild bacterial infection of the skin. The formulations may be specific chemical mixtures to be used as topical skin ointments. The compositions may be used in a skin protectant barrier cream as well as a treatment for dermatitis.

Preferred embodiments may be in the form of an ointment, used for the prevention and/or treatment of dermatitis, including diaper dermatitis and/or dermatitis associated with candida and/or mild bacterial skin infections.

Some embodiments may include topical ointments for the treatment of dermatitis in other anatomical areas, including but not limited to, skin folds, underneath the breasts, and/or in the inguinal region.

Some embodiments may be used as a topical therapy for eczema, psoriasis and/or any other inflammatory condition of the skin.

### BRIEF DESCRIPTION OF THE DRAWINGS

The present invention is illustrated and described herein with reference to the drawings, in which:

FIG. 1 is a diagram of an example embodiment of a topical composition as applied to the skin's surface, in accordance with the present disclosure.

### DETAILED DESCRIPTION OF THE DISCLOSURE

The presently disclosed compositions are unique and innovative due to differing active ingredients and methods of action. The presently disclosed compositions take a different, multifaceted approach, unlike the prior art, in solving the problem of dermatitis, specifically diaper dermatitis.

Disclosed herein are compositions and methods for the prevention and treatment of dermatitis. The pharmacological compositions provide a multifaceted approach to the healing and prevention of dermatitis conditions, including “diaper rash” or “diaper dermatitis”.

Using miconazole, to control yeast overgrowth on the skin, and/or bacitracin, to control bacteria on the skin, has not previously been combined with the use of the disclosed skin protectants.

The disclosed compositions may be easy to use, easy to wash off, inexpensive to manufacture, as well as safe and effective alone or in combination with other established treatments for these skin disorders.

The present disclosure provides improved compositions for skin protection and treatment of general dermatitis and diaper dermatitis or “diaper rash”.

The present disclosure provides improved compositions for the treatment of rashes which may be caused by yeast and/or bacteria.

The present disclosure provides novel uses for a combination of bacitracin, miconazole, aluminum hydroxide and a skin protectant ointment.

Multifaceted approaches to treating dermatitis may include eliminating the sources of the irritation, soothing the irritated skin, and preventing further infections. This could further entail keeping the surface of the skin from getting overly hydrated through ensuring dryness.

Barrier emollients may be used to keep moisture and irritants away from an infant's delicate skin. Skin protectants may help prevent contact between skin and irritants. Conventional skin protectants include about 10-40% zinc oxide mixed with an oil base, which emits an unpleasant odor. Some prior products incorporated alkoxylated ether/esters to lessen the greasy consistency.

The compositions may be effective topical protectants for preventing contact between the skin and external irritants. The skin protectant ingredients may be hydrophobic to repel external moisture and protect the skin from hyperhydration.

The compositions may be formulated as ointments or water-in-oil emulsions may have a high viscosity that helps it stick to the skin, thereby providing an effective barrier for the skin.

The compositions may have emollient properties for conditioning, moisturizing, soothing, and/or softening the skin.

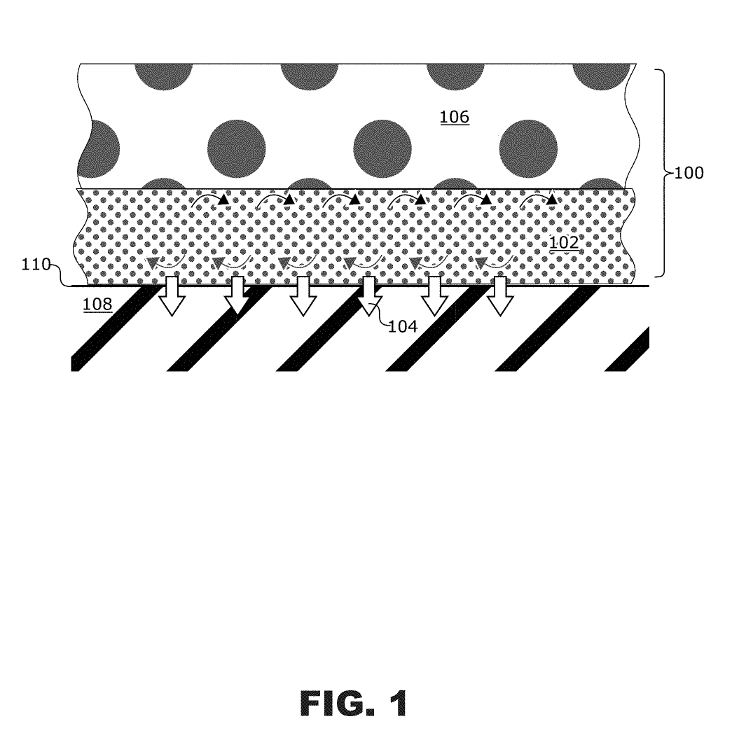

As shown in FIG. 1, a topical composition 100 may be applied to the skin 108 of a patient. The topical composition 100 may include an emollient agent 104 that is absorbed through the surface 110 of the skin 108 to moisturize, condition, and/or otherwise soothe the skin 108 that may be suffering from some form of dermatitis or other skin irritation. The topical composition 100 may further include a desiccant agent 102 that wicks away water from the surface 110 of the skin 108. Finally, the topical composition 100 may include a skin protectant 106. The skin protectant 106 may function to seal in moisture for the skin 108 to prevent over drying. Moreover, the skin protectant 106 may function as a barrier to prevent contact between external irritants and the skin 108.

Petroleum has long been recognized as a lubricant and ointment. The semi-solid, petroleum may be known as petroleum jelly, petrolatum, white petrolatum, soft paraffin, paraffin wax, multi-hydrocarbon, or CAS number 8009-03-8. The complex hydrocarbons of petroleum may include aliphatic, alicyclic and aromatic hydrocarbons. The ingredient petrolatum may be used as a skin protectant in drug products. Petrolatum may be applied as needed. The ingredient petrolatum may serve multi-purpose uses in over-the-counter drug products. Petrolatum is generally recognized as safe and effective in over-the-counter drug protectant products.

Lanolin may be used as a protectant or emollient active ingredient. Over-the-counter skin protectant diaper rash drug products may use about 15.5% lanolin as a skin protectant active ingredient in combination with other skin protectant active ingredients for the treatment and prevention of diaper rash. Anhydrous lanolin may be included at a 1-10% concentration in combination with one or more oleaginous emollients. In over-the-counter anorectal drug products, lanolin may be included as a protectant active ingredient at concentrations of 50% and above as a single ingredient or between 12.5% and 50% in combinations. Lanolin has been marketed as a skin protectant both as a single ingredient and in combination with other ingredients. The concentration in single ingredient products may range from about 37% to 50%. The use concentration may be about 12.5% to 50% in accord with the concentration of marketed single ingredient skin protectant drug products and the concentration used in anorectal protectant combination drug products.

Miconazole or miconazole nitrate is a topical antifungal agent used to treat yeast infections. Miconazole may prevent the ability of fungi to generate cell wall membranes. Specifically, miconazole inhibits the enzyme 14a-sterol demethylase, thereby reducing the production of ergosterol needed to build cell walls. Thus, it may help eliminate a yeast infection of the skin. However, the use of miconazole on its own may cause a rash or irritation.

Aluminum hydroxide, Al(OH)₃, AlH₃O₃, may also be known as aluminum trihydroxide, aluminium(3+) trioxidanide, aluminium(III) hydroxide, aluminium(3+) hydroxide, aluminic acid, aluminic hydroxide, hydrated alumina, orthoaluminic acid, aldrox, algeldrate, alhydrogel, alugel, amphojel, basalgel, brasivil, dialume, nephrox, pepsamer, and rocgel. Aluminum hydroxide may be found in the mineral form gibbsite, γ-Al(OH)₃, α-Al(OH)₃, hydrargillite, or hydrargyllite, as well as other crystalline structures, such as bayerite, β-Al(OH)₃, doyleite, and nordstrandite. Aluminum hydroxide may be in the form of a gel. Aluminum hydroxide powder is soluble in acids. After undergoing a heating process, aluminum hydroxide powder becomes activated alumina, which may be used as a desiccant.

Because aluminum hydroxide is amphoteric, it may function as a pH balancing agent. The composition may include aluminum hydroxide as a pH buffering agent to maintain the pH level of the skin within the normal range, which is from about 4 to about 5.5.

The composition may be an over-the-counter skin protectant drug product containing mineral oil. The ingredient's physical properties may help the effectiveness of mineral oil in skin protectants for diaper rash. Mineral oil may aid in the easy removal of petrolatum.

In some embodiments, the composition may be made of the following ingredients: Petrolatum 0-99.9%, Lanolin 0-16%, Aluminum hydroxide 0-40 mg/g, and Mineral oil 0-5%.

Table A below lays out the possible concentration ranges for the topical composition ingredients in some preferred embodiments.

TABLE A
COMPOSITION INGREDIENT RANGES

| Ingredient | Weight Proportion (%) |
|---|---|
| Petrolatum | up to ~99.9 |
| Lanolin | up to ~16 |
| Miconazole | up to ~1 |
| Aluminum Hydroxide | up to ~4 |
| Mineral Oil | up to ~5 |

Additionally, bacitracin may be included in the composition in amounts up to 500 U/g. Bacitracin is a polypeptide antibiotic secreted from the bacteria Bacillus subtilis var. Tracy. The bacitracin may act as an antibacterial agent within the composition due to the polypeptides in the antibiotic targeting the cell walls of Gram-positive bacteria, such as Staphylococcus aureus, Staphylococcus epidermidis, and Streptococcus pyogenes, for example. Conventionally, bacitracin has been thought to be a skin irritant, however, it may act synergistically with the other ingredients within the presently disclosed composition to help stave off rashes or dermatitis, which may be caused by bacteria.

The formulation of pharmaceutical preparations (e.g., cosmetics, creams, balms) may be as an oil-in-water emulsion. Alternatively, the compositions may be anhydrous compound, containing no water. The compositions may include lipase or other enzyme inhibitors, such as water soluble metallic salts (e.g., zinc chloride).

The compositions may further include an humectant, such as glycerin, lecithin, and/or propylene glycol, for example.

Normally, the pH level of skin is between about 4 and about 5.5. The normal pH of infant skin may be higher than that of adult skin (4.5-7.4). Diaper dermatitis is thought to be caused in part by the skin's contact with urine and/or fecal matter. In particular, urea contained in urine may be broken down into ammonium hydroxide, which increases the pH level in the affected area. Higher pH levels may increase the activity of enzymes (e.g., ureases, lipases, and proteases), which in turn may attack the skin.

While urine may increase the pH level of the skin in the affected area, fecal matter present may lower the pH level due to bile, etc. Either may alter the skin's pH level outside of the normal range, which is from about 4 to about 5.5.

The compositions may advantageously include pH buffering agents or pH buffers, such as aluminum hydroxide, to maintain the pH level of the skin within the normal range.

When the protective barrier of the skin is damaged, it is more permeable to irritants. Moreover, skin that is over-exposed to moisture is made also made more permeable through hyperhydration. Each of these sources of damage and irritation may work simultaneously to further exacerbate dermatitis. Some dermatitis conditions may be caused by a Candida albicans or other yeast infection/overgrowth of the skin. Some dermatitis conditions may be caused by allergic reactions. Some dermatitis conditions may be caused by an infection of the skin and/or hair follicles due to bacteria, such as Streptococcus or Staphylococcus aureus. With all these possible contributing causes of dermatitis, the condition often has an unknown or multifactorial etiology.

Thus, for dermatitis caused by multifactorial and/or unknown etiology, treatment using the disclosed compositions may be simple, as they treat multiple causes at once.

The topical compositions may wash off easily, be inexpensive to manufacture, and be safe and effective alone or in combination with other established treatments for these skin disorders.

In some embodiments, the composition may include magnesium hydroxide, in weight proportions up to about 40%, balanced with the aluminum hydroxide.

In some embodiments, the compositions may be in the form of an ointment used as a skin protectant.

In some embodiments, the compositions may be in the form of an ointment for the treatment of diaper dermatitis of unknown etiology.

In some embodiments, the compositions may be in the form of an ointment for the treatment of diaper dermatitis due to a Candida or other yeast infection of the skin.

In some embodiments, the compositions may be in the form of an ointment for the treatment of diaper dermatitis due to allergic reaction.

In some embodiments, the compositions may be in the form of an ointment for the treatment of diaper dermatitis related to mild infection of the skin or hair follicles by common bacteria that are considered native to the human skin such as Streptococcus.

In some embodiments, the compositions may be in the form of an ointment for the treatment of general dermatitis of unknown etiology.

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are contemplated thereby.

What is claimed is:

1. A pharmacological composition for treating and preventing dermatitis, the composition comprising:
therapeutically effective amounts of:
a skin protectant agent,
an emollient agent, and
a desiccant agent;
in a vehicle for topical application to the skin of a mammal,
wherein the composition wicks moisture from the skin's surface.

2. The composition of claim 1, further comprising an antibacterial agent.

3. The composition of claim 2, wherein the antibacterial agent is miconazole.

4. The composition of claim 1, further comprising an antifungal agent.

5. The composition of claim 4, wherein the antifungal agent is bacitracin.

6. The composition of claim 1, wherein the skin protectant agent is petrolatum.

7. The composition of claim 1, wherein the emollient agent is lanolin.

8. The composition of claim 1, wherein the emollient agent is mineral oil.

9. The composition of claim 1, wherein the emollient agent is aluminum hydroxide.

10. A method of treating and preventing dermatitis comprising:
topically applying a composition including:
petrolatum,
aluminum hydroxide, and
an antibacterial agent.

11. The method of claim 10, wherein the antibacterial agent is miconazole.

12. The method of claim 10, wherein the antibacterial agent is bacitracin.

13. The method of claim 10, wherein the composition further includes lanolin.

14. The method of claim 10, wherein the composition further includes mineral oil.

15. The method of claim 10, wherein the aluminum hydroxide comprises a weight proportion between about 1% and about 4%.

16. An composition comprising:
therapeutically effective amounts of:
a skin protectant agent,
an emollient agent, and
a pH buffering agent;
in a vehicle for topical application to the skin of a mammal,
wherein the composition is substantially free of zinc oxide.

17. The composition of claim 16, wherein the skin protectant agent is aluminum hydroxide gel.

18. The composition of claim 16, wherein the skin protectant agent is petrolatum.

19. The composition of claim 16, wherein the pH buffering agent is aluminum hydroxide.

20. The composition of claim 16, wherein the emollient agent is at least one of lanolin and mineral oil.
